(19) United States
(12) Patent Application Publication
(10) Pub. No.: US 2003/0187493 A1
(43) Pub. Date: Oct. 2, 2003

(54) Coated stent with protective assembly and method of using same

(76) Inventors: Todd Campbell, Petaluma, CA (US); Marvin Cervantes, Santa Rosa, CA (US)

Correspondence Address: MEDTRONIC AVE, INC., 3576 UNOCAL PLACE, SANTA ROSA, CA, 95403, US

(21) Appl. No.: 10/112,146

(22) Filed: Mar. 29, 2002

(51) Int. Cl. A61F 2/06

(52) U.S. Cl. 623/1.11; 623/1.42

(57) ABSTRACT

A stent with a protective assembly is provided. The stent comprises at least one stent segment, operatively adapted for deployment from the sheath member, and at least one sheath member removably enclosing the stent segment and operatively adapted to protect the stent segment from handling. Methods and systems for use of the stent are also provided.

### FIELD OF THE INVENTION

### BACKGROUND OF THE INVENTION

Cardiovascular disease, including atherosclerosis, is the leading cause of death in the U.S. A number of methods and devices for treating coronary heart disease have been developed, some of which are specifically designed to treat the complications resulting from atherosclerosis and other forms of coronary arterial narrowing.

One method for treating atherosclerosis and other forms of coronary narrowing is percutaneous transluminal coronary angioplasty, hereinafter referred to as “angioplasty” or “PTCA”. More than one-third of heart disease patients undergo angioplasty—about 1 million people annually worldwide. Some patients undergo angioplasty repeatedly.

The objective in angioplasty is to enlarge the lumen of the affected coronary artery by radial hydraulic expansion. This is generally accomplished by inflating a balloon within the narrowed lumen of the affected artery. Radial expansion of the coronary artery may occur in several different dimensions, and is related to the nature of the plaque. Soft, fatty plaque deposits are flattened by the balloon, while hardened deposits are cracked and split to enlarge the lumen. The wall of the artery itself may also be stretched as the balloon is inflated.

With simple angioplasty, the balloon is threaded through the artery with a catheter and inflated at the place where the blood vessel is blocked. After the procedure, the balloon is then removed. With simple angioplasty alone, about 40-50 percent of arteries close up again or re-narrow. This narrowing is known as restenosis.

To reduce the risk of restenosis, a stent may also be inserted during angioplasty. The stent may be used to prop open the artery once the balloon is removed. The use of a stent may reduce the risk of restenosis to 20-30 percent. The stent is designed to support plaque damaged arterial walls after a blockage has been removed.

Typically, if restenosis occurs with a stent, the doctors may insert highly radioactive pellets into the artery to help prevent further clogging. This radiation therapy can halve the risk of restenosis but presents all the risks associated with radiation therapy.

Restenosis occurs because the blood vessel wall is injured when the stent is implanted. The area then becomes inflamed and new cells form scar tissue. The arterial walls may become so thick in some instances that they protrude into the mesh of the stent. In such cases, a further angioplasty may be undergone, and a new stent may be placed inside the existing one. If restenosis continues, the eventual alternative may then be bypass surgery.

Alternatively, a coated stent may be inserted during the angioplasty. Such a coated stent may eliminate the need for repeat angioplasties and could spare some patients the trauma, risk and prolonged recovery associated with heart bypass surgery.

The coated stent may be coated, for example, with Rapamune, generically known as sirolimus or rapamycin. This drug is used to prevent organ rejection in kidney transplants. It stops new cells from forming without impairing the healing of the vessel. It also dampens inflammation and has antibiotic properties.

In clinical studies, patients who have received the coated stent do not show this re-narrowing and re-blockage of arteries.

However, because the coating of the stent comprises a therapeutic drug, coated stents present problems associated with drug administration. For example, for a drug to be administered effectively, the integrity of the drug's effective dosage should be maintained. Additionally, contamination of the drug should be avoided. Moreover, certain drugs require regulated conditions for efficacy, such as regulated air circulation or lack thereof, regulated exposure to light, etc.

Currently stents may be protected with a sheath that closely surrounds the stent. With a coated stent, this protective sheath may damage the coating while the sheath is being placed on or removed from the stent. If the sheath is too tight, the coating may stick to the sheath rather than the stent. If the sheath is removed improperly, some of the coating may also be removed. In any of these cases, the dosage of the drug will be reduced.

Additionally, stents are usually introduced via a catheter introducer. While the stent is traversing the introducer, the coating may be removed due to contact with the introducer. Additionally, the stent may absorb materials from the introducer, thereby damaging the coating.

In addition, stents may be sterilized or otherwise treated prior to deployment. Such treatments may also damage the coating.

It would be desirable therefore to provide a protective assembly for a coated stent that overcomes the above.

### SUMMARY OF THE INVENTION

One embodiment of the present invention provides a stent with protective assembly, including at least one stent segment, operatively adapted for deployment from the sheath member and at least one sheath member removably enclosing the stent segment, the sheath member operatively adapted to protect the stent segment from handling. The stent may also include a coating disposed on the at least one stent segment which may comprise one or more of the following: thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, antisecretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, peptides, proteins, enzymes, extracellular matrix components, inhibitors, free radical scavengers, chelators, antioxidants, antipolymerases, antiviral agents, photodynamic therapy agents, gene therapy agents, and conjugates thereof.

In addition, the stent may comprise a catheter operatively adapted to carry the stent segment as well as an expandable balloon portion attached to the catheter operatively adapted to expand an inner lumen of the stent segment. The stent may also comprise at least one retainer ring disposed on the catheter.

The stent may also comprise an introducer operatively adapted to receive the stent segment. The introducer may be adapted to open the at least one sheath member. The introducer may also be adapted to open a sealing assembly used to seal the sheath member. The introducer may also be adapted to receive the sheath member and to retain the sheath member in a first position while the stent is deployed.

The sheath member may comprises two snap-together components and the introducer may be adapted to dehisce the two snap-together components from each other. The sheath member may also be a rigid cone.

The stent may also comprise a seal for sealing the sheath member. An introducer may be used to open the seal. The seal may be adapted to hold an inert gas, such as argon or nitrogen, within the sheath member. The seal may be made of foil. The seal may also be at least one retainer ring disposed on the catheter. The seal may also comprise at least one protrusion operatively attached to the seal, the protrusion operatively adapted to hold the catheter immobile.

Another embodiment of the present invention provides a system for treating heart disease that includes a catheter, a stent coupled to the catheter, the stent including a coating and a sheath removably enclosing the stent. The coating may be a polymer coating and at least one therapeutic agent may be dispersed within the coating or within the stent.

The system may also include an expandable balloon portion operatively attached to the catheter as well as at least one retainer disposed upon the catheter. The system may further include an introducer operatively adapted to receive the stent. Alternatively, the introducer may be adapted to retain the sheath in a first position while the stent is deployed. Alternatively, the introducer may be adapted to release the stent from the sheath.

The system may also include a sealing assembly, operatively adapted to seal the sheath. The sealing assembly may be used to maintain an environment within the seal. Alternatively, the system may include at least one retainer disposed upon the catheter, operatively adapted to seal the sheath.

Another embodiment of the invention provides an introducer for a coated stent, comprising a body portion for receiving the coated stent and an introducer interface disposed on an end of the body portion, the interface operatively adapted to open a sheath. The sheath may be used to enclose the coated stent.

Another embodiment of the invention provides a method for introducing a stent to a target site. An introducer is interfaced with a sheath that is operatively adapted to enclose the stent. The stent is advanced through the introducer via the sheath so that the stent enters the introducer without handling of the stent.

If the stent comprises a coating, the method may further comprise advancing the stent through the introducer via the sheath so that the stent enters the introducer without disturbing the coating.

The stent may be advanced to the target site via a guiding catheter or via a guide wire.

If the stent is disposed upon an expandable balloon portion, the method may also comprise inflating the expandable balloon portion at the target site.

The method may also comprise removing the sheath from the coated stent. The method may also comprise opening the sheath with the introducer. The method may also comprise fastening the sheath within the introducer.

The foregoing, and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims in equivalence thereof.

### BRIEF DESCRIPTION OF THE DRAWINGS

FIG. 1 is a schematic view of one embodiment of a coated stent with a protective assembly in an undeployed configuration in accordance with the present invention;

FIG. 2 is a schematic view of the embodiment of the protective assembly shown in FIG. 1 as the stent is being deployed;

FIG. 3 is a schematic view of the embodiment of FIG. 1 in a deployed configuration in accordance with the present invention;

FIG. 4 is a schematic view of another embodiment of a stent with a protective assembly in accordance with the present invention;

FIG. 5 is a schematic view of yet another embodiment of a stent with a protective assembly in accordance with the present invention;

FIG. 6 is a schematic view of yet another embodiment of a coated stent with a protective assembly in an undeployed configuration in accordance with the present invention;

FIG. 7 is a schematic view of the embodiment of the coated stent with a protective assembly shown in FIG. 6 in an undeployed configuration;

FIG. 8 is a schematic view of the embodiment of FIG. 6 as it is being deployed in accordance with the present invention; and

FIG. 9 is a schematic view of yet another embodiment of a coated stent with a protective assembly in accordance with the present invention.

### DETAILED DESCRIPTION OF THE PRESENTLY PREFERRED EMBODIMENTS

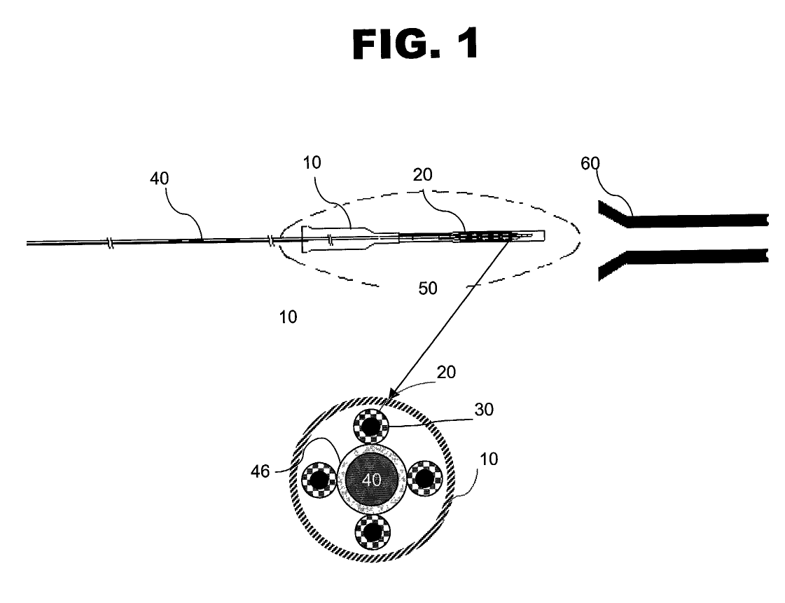

FIG. 1 shows one embodiment of a stent assembly in an undeployed configuration in accordance with the present invention at 50. In one embodiment of the invention, this may be the configuration of stent assembly 50 before it is introduced through a catheter introducer 60. Alternatively, this may be the configuration of stent assembly 50 before sheath 10 is removed in order to expose stent 20 for deployment to the treatment site. Stent assembly 50 may comprise a stent 20 disposed within a sheath 10. In one embodiment of the invention, a coating 30 may be dispersed on stent 20. Alternatively, stent 20 may be any suitable stent requiring a protective assembly, with or without a coating. For example, stent 20 may be a stent formulated of a material requiring a protective assembly. In the embodiment shown in FIG. 1, the stent 20 may be deployed upon a balloon catheter 40. Balloon catheter 40 may further comprise an expandable balloon portion 46.

FIG. 1 further shows a cross-section of stent 20 with coating 30 dispersed thereon. The stent 20 is deployed upon expandable balloon portion 46 of catheter 40. The entire assembly is enclosed within sheath 10. As is seen from the cross section, sheath 10 encloses but does not touch the coating 30 of stent 20. As seen in FIG. 1, sheath 10 may be of sufficient length for entering the entire length of the catheter introducer 60. Thus, the stent 20 is shielded from potential contamination that might occur within the introducer. In addition, the stent 20 may be shielded by sheath 10 such that introducer 60 does not scrape or otherwise disturb coating 30. In an embodiment of the invention where stent 20 is not coated but may be fabricated of a fragile material requiring protection, sheath 10 may also protect stent 20 from contact with introducer 60.

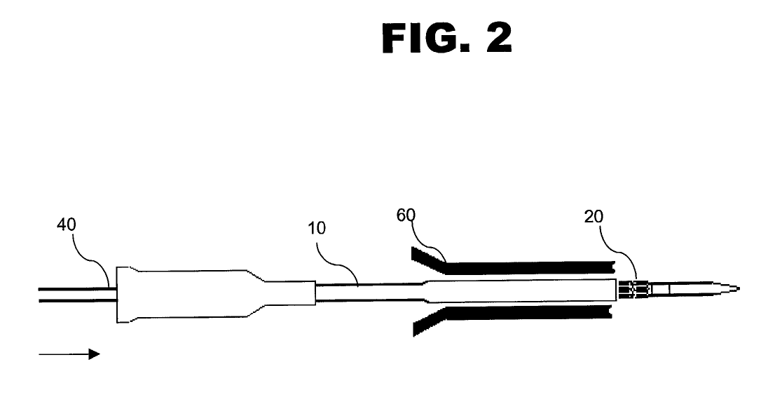

FIG. 2 shows one embodiment of the sheath 10 of stent assembly 50. In one embodiment of the invention, sheath 10 is fabricated of clear material to allow for viewing. Alternatively, the sheath 10 may be fabricated of opaque material to aid in preventing degradation of the coating 30 from light. Alternatively, sheath 10 may be fabricated of ultraviolet-filtering material for protection from UV radiation. The sheath 10 may be made, for example, of Teflon or other suitable materials. As seen in FIG. 2, sheath 10 protects stent 20 from manual handling as stent 20 may be threaded out of sheath 10 by deploying catheter 40 in the direction of the target site (indicated by arrow). Catheter 40 may protrude distally beyond sheath 10 thereby providing a means for manipulating stent 20 without handling the stent within sheath 10. Because stent segment 20 is disposed within sheath 10, sheath 10 may be handled without handling stent segment 20. Stent segment 20 is therefore, less likely to be disturbed during storage, handling or sheath placement. Furthermore, sheath 10 may be handled without disturbing a coating 30 that may be dispersed on stent segment 20. Coating 30 is therefore, less likely to be disturbed during storage, sheath placement and during lab handling.

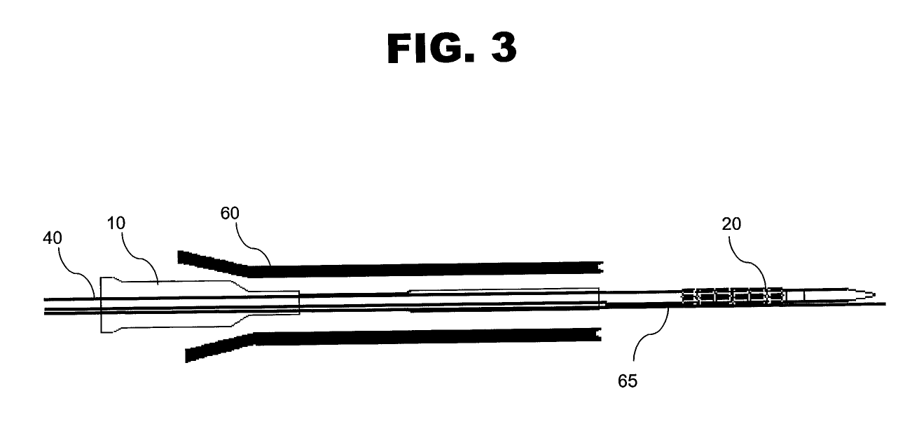

FIG. 3 shows one embodiment of a stent assembly in a deployed configuration in accordance with the present invention. In one embodiment of the invention, this may be the configuration of stent assembly 50 after it has been introduced through a catheter introducer 60. Alternatively, this may be the configuration of stent assembly 50 after sheath 10 has been removed in order to expose stent 20 for deployment to the surgical site. In this configuration, once sheath 10 has been removed to expose stent 20, stent 20 may be used as is well known in the art. For example, as seen in FIG. 3, once the stent 20 has been deployed on catheter 40 through the length of introducer 60, sheath 10 remains behind in the introducer 60. Meanwhile, stent 20 continues along guide wire 65 for deployment to a target treatment site as is well known in the art.

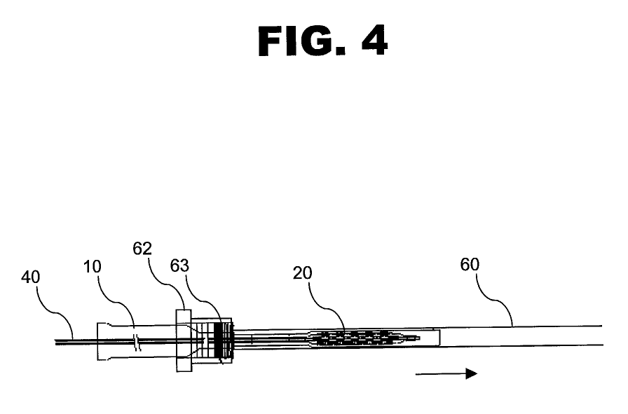

FIG. 4 shows another embodiment of a stent assembly in a deployed configuration in accordance with the present invention. In one embodiment of the invention, catheter introducer 60 is a modified introducer comprising toughy 62 and O-ring 63. As catheter 40 is played out to push stent 20 forward through the introducer 60 (in the direction of the target site as indicated by the arrow), toughy 62 may be tightened to immobilize sheath 10. Sheath 10 may thus remain attached to the introducer 60. Meanwhile, stent segment 20 passes unimpeded through the introducer 60 along the catheter 40 as further described below.

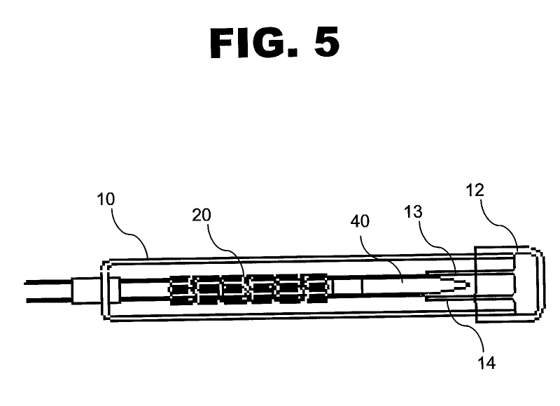

In yet another embodiment of the invention, as illustrated in FIG. 5, sheath 10 may further comprise a sealing assembly 12. This sealing assembly may be, for example, a cap or seal to provide further protection to stent segment 20. Sealing assembly 12 may be constructed of the same material as sheath 10. Sealing assembly 12 may be made, for example, of Teflon, of foil or other suitable materials. In one embodiment of the invention, sealing assembly 12 is fabricated of clear material to allow for viewing. Alternatively, sealing assembly 12 may be fabricated of opaque material to aid in preventing degradation of the coating 30 from light. Alternatively, sealing assembly 12 may be fabricated of ultraviolet-filtering material for protection from UV radiation. As seen in FIG. 5, sealing assembly 12 may further comprise protrusions 13, 14. These protrusions may be used to hold catheter 40 immobile within sheath 10.

Sheath 10 combined with sealing assembly 12 may be used in any suitable manner to preserve a desired environment within sheath 10 for stent segment 20 and/or coating 30. For example, the use of sealing assembly 12 may enable one or more inert gases, such as argon or nitrogen, to be held within sheath 10. These inert gases may be used to prevent oxidation of the polymers that may comprise coating 30 and/or of the therapeutic agents that may comprise coating 30. Alternatively, these inert gases may be used to prevent oxidation of an uncoated stent 20 formed of a sensitive material.

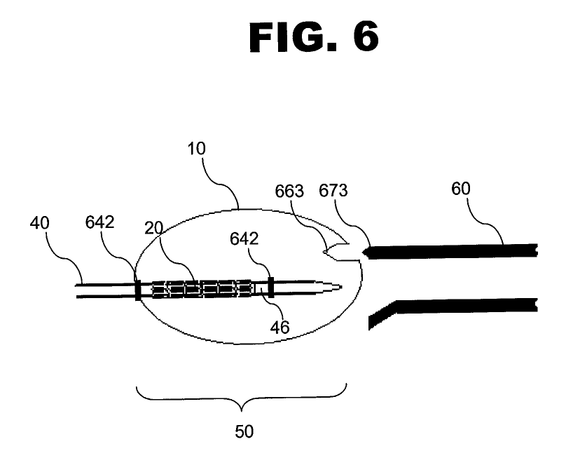

As seen in FIG. 6, in another embodiment of the invention, sheath 10 may be a two-piece snap together sheath that is substantially larger in diameter than the diameter of stent 20. FIG. 6 shows the configuration of stent assembly 50 before it is introduced through catheter introducer 60. In one embodiment of the invention, a coating 30 may be dispersed on stent 20. In the embodiment shown in FIG. 6, the stent 20 may be deployed upon a balloon catheter 40. Balloon catheter 40 may further comprise an expandable balloon portion 46.

As seen in FIG. 6, some embodiments of the invention may include stent retainer rings 642 at each end of the stent 20 to help to maintain the stent on the balloon. These retainers 642 may be located at the proximal and/or distal end of the balloon. Such retainers may be located on top of the balloon 46 or within the balloon 46. Additionally, the balloon portion 46 itself may be used to form one or more stent retainers during encapsulation. Retainers 642 may assist in delivery by providing a smooth transition between the encapsulated stent and the catheter surface. In the embodiment shown in FIG. 6, retainers 642 may also be used to help maintain sheath 10 in its position along catheter 40. Alternatively, retainers 642 may be used to help maintain stent 20 within sheath 10. Alternatively, retainers 642 may serve as a sealing assembly to seal sheath 10. Sheath 10, when sealed using one or more retainers 642, may be used in any suitable manner to preserve a desired environment within sheath 10 for stent segment 20 and/or coating 30. For example, one or more inert gases, such as argon or nitrogen, may be held within sheath 10. These inert gases may be used to prevent oxidation of the polymers that may comprise coating 30 and/or of the therapeutic agents that may comprise coating 30. Alternatively, these inert gases may be used to prevent oxidation of an uncoated stent 20 formed of a sensitive material.

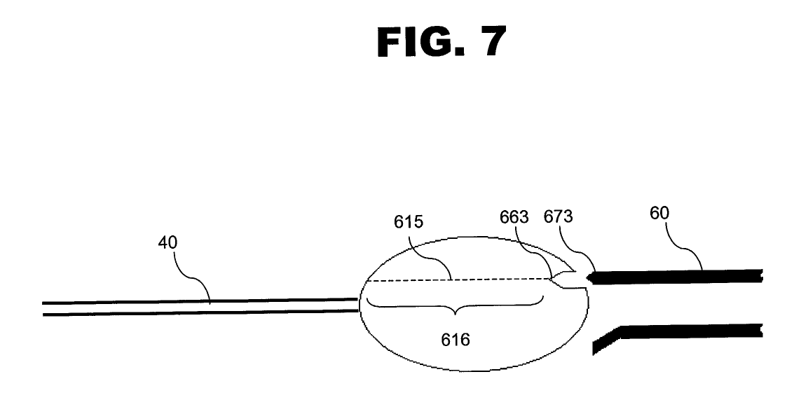

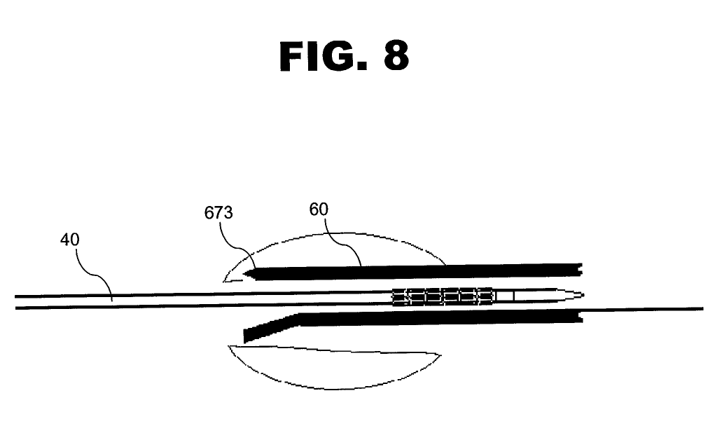

FIG. 7 shows the outside of the embodiment of sheath 10 seen in FIG. 6. As seen in FIG. 7, in one embodiment of the invention, the sheath 10 includes perforations 615. These perforations may allow venting of the stent 20 and/or of coating 30. In addition, the perforations 615 may create a seam 616, along which sheath 10 may be opened as it is introduced through introducer 60. This is best seen in FIG. 8. As seen in FIGS. 6-8, introducer 60 may index with the sheath 10 at one or more index points 663. The distal portion of the sheath 10 may open along the seam 616 as the stent 20 is introduced into the introducer 60. Thus, the stent 20 may be inserted without scraping the coating 30 of stent 20. As FIGS. 6-8 show, introducer 60 may be a modified introducer with a sheath interface 673. Interface 673 may be any suitable assembly that enables introducer 60 to open sheath 10 or otherwise access stent 20 within sheath 10. For example, FIGS. 6-8 show interface 673 as a pointed assembly, which can dehisce sheath 10 along seam 616, beginning at index point 663.

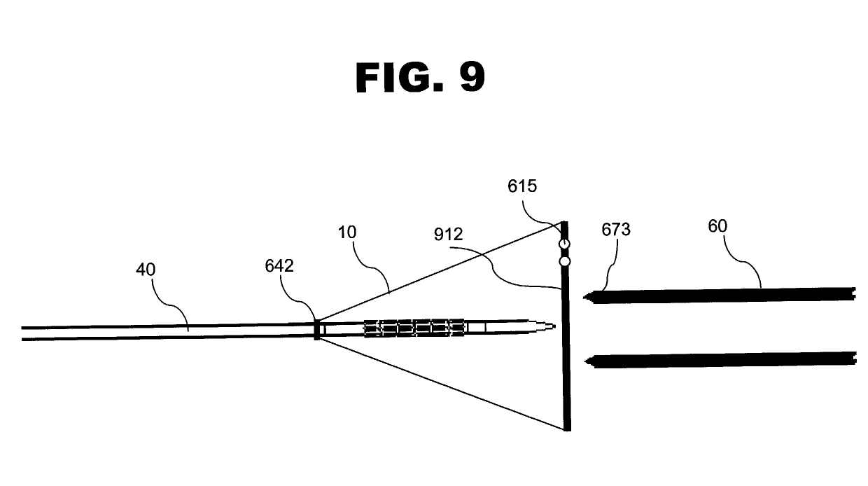

As seen in FIG. 9, in another embodiment of the invention, sheath 10 may be a rigid cone with a seal 912 that may be punctured or otherwise opened. As described above, seal 912 may include perforations 615 that may allow venting of stent segment 20 and/or of coating 30. As seen in FIG. 9, introducer 60 may be a modified introducer with a sheath interface 673 that is adapted to puncture or otherwise open seal 912 of sheath 10. The seal 912 of the sheath 10 may open as the stent 20 is introduced into the introducer 60. Thus, the stent 20 may be inserted without scraping the coating 30 of stent 20. Alternatively, seal 912 may be manually removed before stent 20 is introduced into introducer 60.

As seen in FIG. 9, some embodiments of the invention may include one or more retainer rings 642 at a proximal end of sheath 10. These retainer rings 642 may be used to help maintain the stent on the balloon. In the embodiment shown in FIG. 6, retainers 642 may also be used to help maintain sheath 10 in its position along catheter 40. Alternatively, retainers 642 may be used to help maintain stent 20 within sheath 10. Alternatively, retainers 642 may serve as a sealing assembly to seal sheath 10. Sheath 10, when sealed in combination with retainer 642, may be used in any suitable manner to preserve a desired environment within sheath 10 for stent segment 20 and/or coating 30. For example, one or more inert gases, such as argon or nitrogen, may be held within sheath 10. These inert gases may be used to prevent oxidation of the polymers that may comprise coating 30 and/or of the therapeutic agents that may comprise coating 30. Alternatively, these inert gases may be used to prevent oxidation of an uncoated stent 20 formed of a sensitive material.

Sheath 10 may be fabricated of any suitable material that provides protection of stent 20. For example, sheath 10 may be made of Teflon or other suitable materials. In some embodiments of the invention, sheath 10 is fabricated of clear material to allow for viewing. Alternatively, the sheath 10 may be fabricated of opaque material to aid in preventing degradation of the coating 20 from light. Alternatively, sheath 10 may be fabricated of ultraviolet-filtering material for protection from UV radiation.

In the embodiment shown in FIGS. 1-9, one stent segment 20 is shown. However more stent segments 20 may be used depending upon the size and configuration of the narrowed vessel to be treated. Additionally, when more than one stent segment 20 is used, the segments may be connected together by articulated or rigid joints, or multiple single stent segments may be deployed on the balloon catheter 20. When more than one stent segment 20 is deployed on the catheter 40, each segment may have an associated sheath 10. Alternatively, a plurality of stent segments 20 may be disposed within a sheath 10.

Stent segment 20 may be any suitable device for mechanically keeping an effective blood vessel open after completion of the angioplasty procedure. Such mechanical endoprosthetic devices, which are generally referred to as stents, are typically inserted into the vessel, positioned across the lesion, and then expanded to keep the passageway clear. Stent 20 may be for example, any stent known in the art, including, but not limited to, a coronary stent such as that sold by Medtronic as the S7 system. Stent segment 20 may be used to overcome the natural tendency of the vessel walls of some patients to close back down, thereby maintaining a more normal flow of blood through that vessel than would be possible if the stent were not in place.

Stent segment 20 may be a short, single wire stent having an expandable, generally cylindrical body portion defining an inside surface and an outside surface. Stent segment 20 may comprise a plurality of upper and lower axial turns that permit the stent segment 20 to be compressed or expanded over a wide range while still maintaining a significant mechanical force, such as required to prevent a vessel from restenosis or recoiling.

Stent segment 20 may be constructed of any suitable implantable materials having good mechanical strength. For example, stent segment 20 may be constructed of implantable quality stainless steel or the alloy MP35N. Alternatively, stent segment 20 may be constructed of any other suitable metallic, plastic material, including biodegradable materials. The outside of the stent segment 20 may be selectively plated with platinum, or other implantable radiopaque substances, to provide improved visibility during fluoroscopy. The cross-sectional shape of the finished stent segment 20 may be circular, ellipsoidal, rectangular, hexagonal, square, or other polygon.

The minimum length of each stent segment 20, or the distance between the upper and lower axial turns may be determined based on the size of the vessel into which the stent 20 will be implanted. If more than one stent segment 20 is used, the stent segments 20 may be connected together by articulated or rigid joints, or they may be deployed in a multiple spaced apart, non-connected configuration. Stent segments 20 may be of sufficient length as to maintain its axial orientation with the vessel without shifting under the hydraulics of blood flow (or other fluid flow in different types of vessels), while also being long enough to extend across at least a significant portion of the affected area. At the same time, the stent 20 may be short enough as to not introduce unnecessarily large amounts of material as might cause undue thrombosis.

For example, stent segment 20 may be a self-expanding and expandable stent as is known in the art. Stent segment 20 may be a tubular slotted stents. Stent segment 20 may also comprise connected stents, articulated stents, and multiple connected or non-connected stents. In one embodiment of the invention, stent segment 20 may be formed from a single piece of wire defining axial bends or turns between straight segments. Stent segment 20 may be used for example, for PTCA type stenting, graft support, graft delivery, neurovascular use, GI tract use, drug delivery, and biliary stenting. In one embodiment of the invention, after the stent is positioned across the lesion, it is expanded by the delivery device, causing the length of the stent to contract and the diameter to expand. Depending on the materials used in construction of the stent, the stent maintains the new shape either through mechanical force or otherwise.

As seen in FIGS. 6-8, some embodiments of the invention may include stent retainer rings 642 at each end of the stent to help to maintain the stent on the balloon. These retainers may be located at the proximal and/or distal end of the balloon. Such retainers may be located on top of the balloon or within the balloon. Additionally, the balloon portion 46 itself may be used to form one or more stent retainers during encapsulation. Retainers may assist in delivery by providing a smooth transition between the encapsulated stent and the catheter surface. Alternatively, the stent 20 may be retained on the delivery catheter by means of either (a) plastically deforming the stent so that it is crimped onto the balloon, or (b) having the stent exhibit a small enough internal diameter to act as an interference fit with the outside diameter of the balloon catheter.

As seen in FIGS. 1-9, coating 30 may comprise any suitable therapeutic agent for delivering therapy to a target site and/or any suitable substance within which such therapeutic agents may be dispersed. Coating 30 may be a coating adapted to deliver sustained release of therapeutic agent to target cells. Coating 30 may be, for example a biodegradable coating or a porous non-biodegradable coating, having dispersed therein a sustained-release dosage form of one or more therapeutic agents as described below. In an alternative embodiment, a biodegradable stent may also have the therapeutic agent contained therein, i.e., within the stent matrix of stent segment 20. In yet another embodiment of the invention, the therapeutic agent(s) may be within stent segment 20, which is further coated with a coating 30 having the sustained release-dosage form dispersed therein, is also contemplated. This embodiment of the invention would provide a differential release rate of the therapeutic agent, i.e., there would be a faster release of the therapeutic agent from the coating 30 followed by delayed release of the therapeutic agent that was contained in the stent matrix upon degradation of the stent matrix. The stent segment 20 may thus provide a mechanical means of increasing luminal area of a vessel, in addition to providing biological stenting action from the therapeutic agents releasably embedded therein.

Coating 30 may take any suitable form. For example coating 30 may comprise non-degradable microparticulates or nanoparticulates or biodegradable microparticulates or nanoparticulates. The microparticles or nanoparticles may be formed of a polymer-containing matrix that biodegrades by random, nonenzymatic, hydrolytic scission. One embodiment of coating 30 is formed of a mixture of thermoplastic polyesters (e.g., polylactide or polyglycolide) or a copolymer of lactide and glycolide components. The lactide/glycolide structure has the added advantage that biodegradation thereof forms lactic acid and glycolic acid, both normal metabolic products of mammals.

Coating 30 may be, or may comprise a therapeutic substance which inhibits cellular activity at a target site in order to reduce, delay, or eliminate stenosis after angioplasty or other vascular surgical procedures. Coating 30 may also be a conjugate of several therapeutic substances. For example, coating 30 may comprise therapeutic agents that alter cellular metabolism or are inhibitors of protein synthesis, cellular proliferation, or cell migration; therapeutic agents that affect morphology or increases in cell volume; and/or therapeutic agents that inhibit extracellular matrix synthesis or secretion.

In one embodiment, coating 30 may also include a non-cytotoxic therapeutic agent such as, for example, an antisense compound. One example of a non-cytotoxic therapeutic agent is NeuGene® antisense compound, Resten-NG™ (AVI-4126) available form AVI BioPharma, Corvalis, Oregon. Such antisense compounds compete at the mRNA level to block transcription of proteins that are involved in proliferation of the cells that cause restenosis. Antisense compounds may significantly reduce restenosis without prolonging healing times.

In one embodiment, coating 30 may include a cytotoxic therapeutic agent that is a sesquiterpenoid mycotoxin such as a verrucarin or a roridin. Coating 30 may also comprise cytostatic therapeutic agents that inhibit DNA synthesis and proliferation at doses that have a minimal effect on protein synthesis such as protein kinase inhibitors (e.g., staurosporin), suramin, and nitric oxide releasing compounds (e.g., nitroglycerin) or analogs or functional equivalents thereof. In addition, coating 30 may also comprise therapeutic agents that inhibit the contraction or migration of smooth muscle cells and maintain an enlarged luminal area following, for example, angioplasty trauma (e.g., the cytochalasins, such as cytochalasin B, cytochalasin C, cytochalasin D or the like). Coating 30 may also comprise vascular smooth muscle binding proteins that specifically associate with a chondroitin sulfate proteoglycan (CSPG) expressed on the membranes of a vascular smooth muscle cell.

In one embodiment of the invention, coating 30 may comprise agents that exhibit inhibition of a therapeutically significant target cell activity without killing the target cell, or target cell killing activity. For treatment of restenosis of vascular smooth muscle cells, useful therapeutic agents inhibit target cell activity (e.g., proliferation or migration) without killing the target cells. Example therapeutic moieties for this purpose are protein kinase inhibitors (e.g., staurosporin or the like), smooth muscle migration and/or contraction inhibitors (e.g., the cytochalasins, such as cytochalasin B, cytochalasin C, cytochalasin D or the like), suramin, and nitric oxide-releasing compounds, such as nitroglycerin, or analogs or functional equivalents thereof. In cancer therapy, useful therapeutic agents inhibit proliferation or are cytotoxic to the target cells. Example therapeutic moieties for this purpose are Roridin A and Pseudomonas exotoxin, or analogs or functional equivalents thereof. For treatment of immune system-modulated diseases, such as arthritis, useful therapeutic agents deliver cytostatic, cytocidal or metabolism-modulating therapeutic agents to target cells that are accessible by local administration of the dosage form. Example therapeutic moieties for this purpose are Roridin A, Pseudomonas exotoxin, suramin and protein kinase inhibitors (e.g., staurosporin), sphingosine, or analogs or functional equivalents thereof. For treatment of pathologically proliferating normal tissues (e.g., proliferative vitreoretinopathy, corneal pannus and the like), anti-proliferative agents or antimigration agents may be used (e.g., cytochalasins, taxol, somatostatin, somatostatin analogs, N-ethylmaleimide, antisense oligonucleotides and the like).

Other examples of therapeutic agents that may be used alone or in combination within coating 30 include thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti-secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, peptides, proteins, enzymes, extracellular matrix components, inhibitors, free radical scavengers, chelators, antioxidants, antipolymerases, antiviral agents, photodynamic therapy agents, and gene therapy agents.

The dosage of therapeutic agents may be varied depending on the body lumen involved, the result desired, and the therapy indicated. Preferable therapeutic agents are dispersed within the microparticulates or nanoparticulates of coating 30.

The dosage forms of coating 30 may be targeted to a relevant target cell population by a binding protein or peptide. These binding proteins/peptides may be, for example vascular smooth muscle cell binding protein, tumor cell binding protein and immune system effector cell binding protein. Other possible binding peptides include those that localize to intercellular stroma and matrix located between and among vascular smooth muscle cells. Peptides of this type are specifically associated with collagen, reticulum fibers or other intercellular matrix compounds. Tumor cell binding proteins associated with surface cell markers expressed by the target tumor cell population or cytoplasmic epitopes thereof may also be targeted by the present invention. Immune system-modulated target cell binding proteins associated with cell surface markers of the target immune system effector cells or cytoplasmic epitopes thereof may also be targeted with the present invention. The present invention may also be targeted to pathologically proliferating normal tissues.

As seen in FIGS. 1-9, the catheter 40 may be, for example, a low profile design with a tapered distal tip, and an inner lumen for insertion of a conventional guide wire 65. Any conventional or modified balloon. catheter device may be used, such as a PTCA balloon catheter.

As seen in FIGS. 1-9, the balloon portion 46 may be formed from a material such as polyethylene, polyethylene terephthalate (PET), or from nylon or the like. The length and diameter of the balloon may be selected to accommodate the particular configuration of the stent segment 20. The balloon may be carried on any catheter, such as, for example PTCA low profile catheters and over the wire catheters.

The stent assembly 50 may be delivered to the desired site with or without a guiding catheter and using a conventional guidewire for steerability to negotiate the area to be treated. Conventional radiopaque markers and fluoroscopy may be used with the device for positioning the encapsulated stent assembly and for viewing the expansion procedure. Once the stent assembly 50 is in place across the lesion, the balloon may be inflated in a conventional manner. Alternatively, the stent 20 may be a self-inflating assembly which does not require balloon portion 46.

Angioplasty is typically performed as follows: A thin walled hollow guiding catheter is introduced into the body via a relatively large vessel, such as the femoral artery in the groin area or the brachial artery in the arm. Once access to the femoral artery is achieved, a guiding catheter is inserted to maintain a passageway during the procedure. The flexible guiding catheter must negotiate an approximately 180 degree turn through the aortic arch to descend into the aortic cusp where entry may be gained to either the left or the right coronary artery, as desired.

After the guiding catheter is advanced to the area to be treated by angioplasty, a flexible guidewire is inserted into the guiding catheter through an expandable balloon and advanced to the area to be treated. The guidewire is advanced across the lesion, or “wires” the lesion, in preparation for the advancement of balloon catheter 40 having an expandable balloon portion 46 composed of polyethylene, polyvinyl chloride, polyolefin, or other suitable substance, across the guide wire.

As described above, in one embodiment of the invention, sheath 10 is removed just before balloon catheter 40 is introduced through introducer 60. In another embodiment of the invention, sheath 10 is removed as stent assembly 50 is being deployed through catheter introducer 60.

The use of the relatively rigid guide wire is often necessary for steerability to advance the catheter through the narrowed lumen of the artery and to direct the balloon, which is typically quite flexible, across the lesion. Radiopaque markers in the balloon segment 46 of the catheter facilitate positioning across the lesion. The balloon catheter 40 is then inflated with contrast material to permit fluoroscopic viewing during treatment. The balloon is alternately inflated and deflated until the lumen of the artery is satisfactorily enlarged.

The exterior wall of the vessel attempts to return to its original shape through elastic recoil. The stent 20, however, remains in its expanded form within the vessel, and prevents further recoil and restenosis of the vessel. The stent maintains an open passageway through the vessel. Because of the low mass of the preferred support device of the present invention, thrombosis is less likely to occur. Ideally, the displacement of the plaque deposits and the implantation of the stent will result in a relatively smooth inside diameter of the vessel.

While the primary application for the stent is presently believed to be treatment of cardiovascular disease such as atherosclerosis or other forms of coronary narrowing, the stent assembly of the present invention may also be used for treatment of vessels in the kidney, leg, carotid, or elsewhere in the body. In such other vessels, the size of the stent may need to be adjusted to compensate for the differing sizes of the vessel to be treated.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

We claim:

1. A stent with protective assembly, comprising:
at least one stent segment, operatively adapted for deployment from the sheath member; and
at least one sheath member removably enclosing the stent segment, the sheath member operatively adapted to protect the stent segment from handling.

2. The stent of claim 1, further comprising:
a coating disposed on the at least one stent segment.

3. The stent of claim 2, wherein the coating is selected from the group consisting of:
thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, peptides, proteins, enzymes, extracellular matrix components, inhibitors, free radical scavengers, chelators, antioxidants, antipolymerases, antiviral agents, photodynamic therapy agents, gene therapy agents, and conjugates thereof.

4. The stent of claim 1, further comprising:
a catheter operatively adapted to carry the stent segment.

5. The stent of claim 4, further comprising:
an expandable balloon portion operatively adapted to expand an inner lumen of the stent segment, the expandable balloon portion operatively attached to the catheter.

6. The stent of claim 1, further comprising:
an introducer operatively adapted to receive the stent segment.

7. The stent of claim 6 wherein the introducer is operatively adapted to open the at least one sheath member.

8. The stent of claim 6 wherein the introducer is operatively adapted to open a sealing assembly, the sealing assembly operatively adapted to seal the sheath member.

9. The stent of claim 1, further comprising:
an introducer operatively adapted to receive the sheath member.

10. The stent of claim 9 wherein the introducer is operatively adapted to retain the sheath member in a first position while the stent is deployed.

11. The stent of claim 1 wherein the at least one sheath member comprises two snap-together components.

12. The stent of claim 10, further comprising:
an introducer operatively adapted to dehisce the two snap-together components from each other.

13. The stent of claim 1, further comprising:
a seal, operatively adapted to seal the sheath member.

14. The stent of claim 13, further comprising:
an introducer operatively adapted to open the seal.

15. The stent of claim 13 wherein the seal is operatively adapted to hold an inert gas within the sheath member.

14. The stent of claim 13 wherein the inert gas is selected from the group consisting of:
argon and nitrogen.

15. The stent of claim 13 wherein the seal is made of foil.

16. The stent of claim 13 wherein the sheath member is a rigid cone.

17. The stent of claim 13 wherein the seal is at least one retainer ring disposed on the catheter.

18. The stent of claim 13, further comprising:
at least one protrusion operatively attached to the seal, the protrusion operatively adapted to hold the catheter immobile.

19. The stent of claim 1, further comprising:
at least one retainer ring disposed on the catheter.

20. A system for treating heart disease, comprising:
a catheter;
a stent coupled to the catheter, the stent including a coating; and
a sheath removably enclosing the stent.

21. The system of claim 20 wherein the coating is a polymer coating.

22. The system of claim 20, further comprising:
at least one therapeutic agent dispersed within the coating.

23. The system of claim 20, further comprising:
at least one therapeutic agent dispersed within the stent.

24. The system of claim 20, further comprising:
an expandable balloon portion operatively attached to the catheter.

25. The system of claim 20, further comprising:
an introducer operatively adapted to receive the stent.

26. The system of claim 20, further comprising:
an introducer operatively adapted to retain the sheath in a first position while the stent is deployed

27. The system of claim 20, further comprising:
an introducer operatively adapted to release the stent from the sheath.

28. The system of claim 20, further comprising:
a sealing assembly, operatively adapted to seal the sheath.

29. The system of claim 28 wherein the sealing assembly is operatively adapted to maintain an environment within the seal.

30. The system of claim 28, further comprising:
at least one retainer disposed upon the catheter, operatively adapted to seal the sheath.

31. The system of claim 20, further comprising:
at least one retainer disposed upon the catheter.

32. An introducer for a coated stent, comprising:
a body portion for receiving the coated stent, and
an introducer interface disposed on an end of the body portion, the interface operatively adapted to open a sheath.

33. The introducer of claim 32 wherein the sheath is operatively adapted to enclose the coated stent.

34. A method for introducing a stent to a target site, comprising:
interfacing an introducer with a sheath, the sheath operatively adapted to enclose the stent; and
advancing the stent through the introducer via the sheath so that the stent enters the introducer without handling of the stent.

35. The method of claim 34 wherein the stent comprises a coating, further comprising:
advancing the stent through the introducer via the sheath so that the stent enters the introducer without disturbing the coating.

36. The method of claim 34 further comprising:
advancing the stent to the target site via a guiding catheter.

37. The method of claim 34 further comprising:
advancing the stent to the target site via a guide wire.

38. The method of claim 34 wherein the stent is disposed upon an expandable balloon portion

39. The method of claim 38 further comprising:
inflating the expandable balloon portion at the target site.

40. The method of claim 34, further comprising:
removing the sheath from the coated stent.

41. The method of claim 34, further comprising:
opening the sheath with the introducer.

42. The method of claim 34, further comprising:
fastening the sheath within the introducer.
